(19) United States
(12) Patent Application Publication
(10) Pub. No.: US 2026/0232398 A1
(43) Pub. Date: Aug. 13, 2026

(54) EXTENDED REALITY SYSTEM FOR ROBOTIC SURGERY

(71) Applicant: The Chinese University of Hong Kong, Hong Kong (CN)

(72) Inventors: Qi Dou, Hong Kong (CN); Yonghao Long, Hong Kong (CN); Chengkun Li, New Territories, Hong Kong (CN)

(21) Appl. No.: 19/457,379

(22) Filed: Jan. 23, 2026

Related U.S. Application Data
(60) Provisional application No. 63/756,481, filed on Feb. 10, 2025.

(51) Int. Cl. A61B 34/35; A61B 34/00; A61B 90/00; G16H 40/67

(52) U.S. Cl. CPC A61B 34/35; A61B 90/361; G16H 40/67; A61B 2034/252; A61B 2090/365; A61B 2090/367; A61B 2090/373

(57) ABSTRACT

A system for robotic surgery includes a surgical view server including a processor and a memory unit, a surgical console, and a surgeon mentor apparatus remote to the surgical console. The surgical view server wirelessly communicates with the surgical console and surgeon mentor apparatus; receives a video of a surgical scene from a surgical scope; and transmits the video to the surgeon mentor apparatus, which reconstructs the surgical scene as a 3D virtual surgical scene; presents the 3D virtual surgical scene; and transmits surgery guidance received at the surgeon mentor apparatus. The surgical view server receives guidance from the surgeon mentor, overlays the guidance onto the surgical scene from the surgical scope, transmits the overlaid guidance onto the video of the surgical scene for presentation to the surgeon, and the surgical console presents the video of the surgical scene with the guidance overlaid onto the video of the surgical scene.

### TECHNICAL FIELD

The present invention relates to an extended reality system for robotic surgery.

### BACKGROUND

Robot assisted surgery has become more popular in clinical practice. One example is robot assisted minimally invasive surgery, which utilises robotic systems to assist surgeons in performing minimally invasive procedures. Robot assisted surgery has rapidly evolved and brought numerous advantages in the field of surgery.

However surgical training and guidance is facing challenges with limited access to experts by young surgeons, imbalanced regional medical resource distribution and high costs associated with travel for experts to different trainee sites. Remote mentoring which connects mentors and mentees in different locations via the internet has emerged as a solution. Telemonitoring systems are technology that is commonly used. In some instances, telemonitoring systems. However, telemonitoring systems have limitations with bandwidth to accommodate multiple users and utilise 2D displays of a surgeon's view and 2D information overlays from the mentor. A 2D overlay is limited as it cannot provide depth perception to the surgeon.

Surgeons require precise awareness of surgical instrument positions within the patient's body to avoid mechanical damage during surgery. Timely and effective visual feedback is crucial, but traditional methods for providing visual feedback during surgical procedures rely heavily on the computational power of the Head Mounted Device or Head Mounted Display (HMD) itself, which can lead to several limitations. The HMD's processing capabilities can be overwhelmed by the demands of real-time image recognition, resulting in slow rendering of 3D virtual objects, increased latency and delayed updates. This can cause surgeons to experience frequent shifts in focus, leading to potential challenges and errors during the procedure. Additionally, spatial constraints can hinder assisting surgeons, causing line-of-sight obstructions and delayed information synchronization, which can further reduce surgical efficiency.

### SUMMARY OF THE INVENTION

The present invention relates to an extended reality system for to facilitate robotic surgery and improve the robotic surgery experience. The extended reality system provides a system that allows for remote mentoring of a surgeon by a mentor. The extended reality system enables remote mentors to remotely offer guidance to surgeons performing surgery. The system described herein further includes a distributed computing architecture where complex processing is offloaded to a separate computing device coupled to a head mounted device (i.e., a head mounted display) associated with the mentor. The system described herein addresses at least some of the problems described earlier or provides the public with a useful alternative.

In accordance with a first aspect, the present invention provides an extended reality system for robotic surgery comprising:
a surgical view server comprising a processor and a memory unit,
a surgical console adapted for use by a surgeon,
a surgeon mentor apparatus adapted for use by a surgeon mentor that can mentor a surgeon performing a surgery,
wherein the surgeon mentor apparatus is remote to the surgical console,
the surgical view server is configured to wirelessly communicate with the surgical console and surgeon mentor apparatus,
the surgical console and the surgeon mentor apparatus are arranged as clients to the surgical view server,
wherein the surgical view server is configured to:
   receive a video of a surgical scene from a surgical scope,
   transmit the video of the surgical scene to the surgeon mentor apparatus,
wherein the surgeon mentor apparatus configured to:
   reconstruct surgical scene as a 3D virtual surgical scene,
   present the 3D virtual surgical scene,
   transmit surgery guidance received from the surgeon mentor at the surgeon mentor apparatus,
wherein the surgical view server is further configured to:
   receive guidance from the surgeon mentor,
   overlay the guidance from the surgeon mentor onto the surgical scene from the surgical scope,
   transmit the overlaid guidance from the surgeon mentor onto the video of the surgical scene for presentation to the surgeon, and;
wherein the surgical console is configured to:
   present the video of the surgical scene with the guidance overlaid onto the video of the surgical scene.

The system is advantageous because it allows a remote surgeon mentor to provide guidance to a surgeon performing a surgical process in substantially real time. The guidance from the remote surgeon mentor may be overlaid onto the real-world video from a surgical scope to provide real time guidance to a surgeon.

In one example the system comprises a surgical assistant device operatively coupled to the surgical view server and configured to communicate with the surgical view server, the surgical assistant device arranged as a client to the surgical view server,
wherein the surgical assistant device comprises a camera and is configured to capture video of the assistant's view,
wherein the surgical view server is configured to:
   transmit the video of the surgical scene to the surgical assistant,
   receive a surgical assistant's view from the surgical assistant device,
   overlay the surgical assistant's view onto the surgical scene,
   transmit the surgical scene with the overlaid guidance and the overlaid surgical assistant's view to the surgical console for presentation to the surgeon, and
wherein the surgical console is configured to present the video of the surgical scene with the guidance and the surgical assistant's view overlaid onto the video of the surgical scene.

The system is advantageous because the system's architecture allows multiple users to assist the surgeon. The system is advantageous because it allows for multi user collaboration in real time.

In one example the guidance is overlaid in 3D onto the video of the surgical scene. The guidance may be a virtual tool with a movement of the virtual tool indicating to the surgeon how the real-world surgical tool should be manipulated. The guidance is overlaid in 3D allowing for depth perception and a more realistic experience for the surgeon.

In one example the guidance is overlaid onto the video of the surgical scene by stereo occlusion aware overlaying. This is advantageous as it improves depth perception of the surgeon.

In one example the guidance overlaid onto the video the surgical scene comprises one or more of: a virtual surgical tool and direction of motion of the virtual surgical tool, or an arrow or line indicating a motion of a surgical tool being used by the surgeon.

In one example the 3D virtual surgical scene corresponds to a surgical scene visible in the video, and the 3D virtual surgical scene is presented on an interface of the surgical mentor apparatus as an extended reality space.

In one example the surgical view server is configured to:
extract RGB data from the received video,
estimate a depth map from the received video,
combine the RGB data and the depth map into an encoded RGBD data,
serialize the RGBD data to generate a stream of RGBD data,
transmit the serialized RGBD data to the surgical mentor apparatus and the surgical assistant device in real time for 3D reconstruction.

In one example the surgical assistant device and the surgeon mentor apparatus are configured to stream the serialized RGBD data in real time, and;
reconstruct the 3D virtual surgical scene based on the RGBD data and wherein the 3D virtual surgical scene corresponds to the surgical scene captured in the video, and the 3D virtual surgical scene is updated in real time to correspond with the surgical scene captured in the video.

In one example the surgical mentor apparatus comprises:
a head mounted device,
an extended reality server operatively coupled to the head mounted device and configured to communicate with the head mounted device,
wherein the extended reality server is configured to perform 3D scene processing and rendering operations to render the 3D virtual surgical scene and transmit the rendered 3D virtual surgical scene to the head mounted device for presentation to a surgeon mentor.

In one example extended reality server is configured to:
receive the RGBD data from the surgical view server,
decode the received RGBD data,
render the RGBD data as a point cloud,
each point in the point cloud is plotted on a global coordinate frame,
transmit the point cloud to the head mounted device, and;
the head mounted device is configured to render the 3D virtual surgical scene based on the point cloud and global coordinate frame and display the 3D virtual surgical scene a user interface.

According to a second aspect, the present invention relates to an extended reality system for robotic surgery comprising:
a surgical view server comprising a processor and a memory unit,
a surgical console adapted for use by a surgeon,
a surgical assistance device adapted for use by a surgical assistant,
a surgeon mentor apparatus adapted for use by a surgeon mentor that can mentor a surgeon performing a surgery,
wherein the surgeon mentor apparatus is remote to the surgical console,
wherein the surgeon mentor apparatus comprises a head mounted device and an extended reality server configured to communicate with the head mounted device, the extended reality server and the head mounted device are arranged in a distributed computing architecture,
the head mounted device is configured to be function as a client of the extended reality server,
the head mounted device and the
the surgical view server is configured to wirelessly communicate with the surgical console and surgeon mentor apparatus,
the surgical console, the head mounted device, the extended reality server and the surgical assistance device are arranged as clients to the surgical view server,
wherein the surgical view server is configured to:
   receive a video of a surgical scene from a surgical scope,
   extract RGB data from the received video,
   estimate a depth map from the received video,
   combine the RGB data and the depth map into an encoded RGBD data,
   serialize the RGBD data to generate a stream of RGBD data,
   transmit the serialized RGBD data to the surgical mentor apparatus,
wherein the head mounted device is configured to:
   receive the RGBD data,
   transmit the RGBD data to the extended reality server for processing,
wherein the extended reality server is configured to:
   perform image processing on the received RGBD data,
   generate a point cloud from the RGBD data,
wherein the head mounted device is further configured to:
   generate a 3D virtual surgical scene based on the point cloud and global coordinate frame and display the 3D virtual surgical scene a user interface,
wherein the surgical view server is further configured to:
   receive guidance from the surgeon mentor,
   overlay the guidance from the surgeon mentor onto the surgical scene from the surgical scope,
   transmit the overlaid guidance from the surgeon mentor onto the video of the surgical scene for presentation to the surgeon, and;
wherein the surgical console is configured to:
   present the video of the surgical scene with the guidance overlaid onto the video of the surgical scene.

The system is advantageous because it allows a remote surgeon mentor to provide guidance to a surgeon performing a surgical process in substantially real time. The system is further advantageous as it allows collaboration between multiple parties and allows a surgeon to be assisted or trained by multiple parties. The system is scalable and can support multiple additional users.

The system is also advantageous because complex calculations to generate the 3D virtual surgical scene and other complex computations are offloaded to the extended reality server. The head mounted device and the extended reality server may communicate via a PC-head mounted device protocol configured to send data between each other. The extended reality server is advantageous because it is programmed to perform complex calculations to generate the 3D extended reality environment reducing the computing load on the head mounted device, thereby improving speed and reducing latency.

In one example the surgical assistant device comprises a camera and is configured to capture video of the assistant's view,
wherein the surgical view server is configured to:
   transmit the video of the surgical scene to the surgical assistant,
   receive a surgical assistant's view from the surgical assistant device,
   overlay the surgical assistant's view onto the surgical scene,
   transmit the surgical scene with the overlaid guidance and the overlaid surgical assistant's view to the surgical console for presentation to the surgeon, and
wherein the surgical console is configured to present the video of the surgical scene with the guidance and the surgical assistant's view overlaid onto the video of the surgical scene.

In one example the guidance is overlaid in 3D onto the video of the surgical scene and wherein the guidance is overlaid onto the video of the surgical scene by stereo occlusion aware overlaying.

In one example the surgical assistant device and the surgeon mentor apparatus are configured to stream the serialized RGBD data in real time, and; reconstruct the 3D virtual surgical scene based on the RGBD data and wherein the 3D virtual surgical scene corresponds to the surgical scene captured in the video, and the 3D virtual surgical scene is updated in real time to correspond with the surgical scene captured in the video.

In one example the surgical assistant device comprises optical see through head mounted display that comprises a camera.

In one example the surgical view server is configured to:
build a synchronous queue to ensure synchronization of scenarios and operations between the head mounted device and the surgical assistant device,
wherein the synchronization information is classified into status, guidance and a shared view information,
wherein status information represents client device ID, operation status, point cloud visibility and a shared view button,
wherein the guidance information indicates guidance type, guidance position and additional text message,
wherein the share view information relates to a view from a camera of the surgical assistant device and/or the head mounted device, and;
wherein the synchronous queue is configured to reduce the operation collision problem.

In one example the 3D virtual surgical scene corresponds to a surgical scene visible in the video, and the 3D virtual surgical scene is presented as a hologram on an interface of the head mounted device in an extended reality space.

In one example the extended reality server is configured to:
receive the RGBD data from the surgical view server,
decode the received RGBD data,
render the RGBD data as a point cloud,
each point in the point cloud is plotted on a global coordinate frame, and;
transmit the point cloud to the head mounted device,
wherein the extended reality server is further configured to:
generate a virtual surgical tool,
transmit the virtual surgical tool to the head mounted device,
wherein the head mounted device is configured to:
present the virtual surgical tool on the 3D virtual surgical scene,
detect input gestures from the surgeon mentor, wherein the gestures correspond to movements of the virtual surgical tool, and wherein the movements of the virtual surgical tool define the guidance from the surgeon mentor,
transmit the guidance as movements of a surgical tool to the surgical view server for overlaying the guidance onto the video of the surgical scene.

In one example the head mounted device is a Hololens device that is adapted to generate an extended reality interface where the virtual surgical tool can be overlaid on the 3D virtual surgical scene.

In one example the system described herein is configured to render over 300,000 points with a latency of less than 250 ms.

The term “comprising” (and its grammatical variations) as used herein are used in the inclusive sense of “having” or “including” and not in the sense of “consisting only of”.

The term “extended reality environment” means an environment or setting where virtual i.e., digital elements or virtual objects can be added to physical real-world environments. Extended reality environment may include a virtual reality environment, an augmented reality environment or a mixed reality environment.

It is to be understood that, if any prior art information is referred to herein, such reference does not constitute an admission that the information forms a part of the common general knowledge in the art.

### BRIEF DESCRIPTION OF THE DRAWINGS

Embodiments of the present invention will now be described, by way of example, with reference to the accompanying drawings in which:

FIG. 1 illustrates a schematic diagram of an extended reality system for robotic surgery.

FIG. 2 illustrates a real-world implementation of the system of FIG. 1.

FIG. 3 illustrates a schematic diagram of a computing device that may be implemented as a surgical view server and/or an extended reality server of the system in FIG. 1.

FIG. 4 illustrates a system workflow of the extend reality system for robotic surgery.

FIG. 5 illustrates a method of point cloud computing and rendering in shader.

### DETAILED DESCRIPTION OF THE PREFERRED EMBODIMENTS

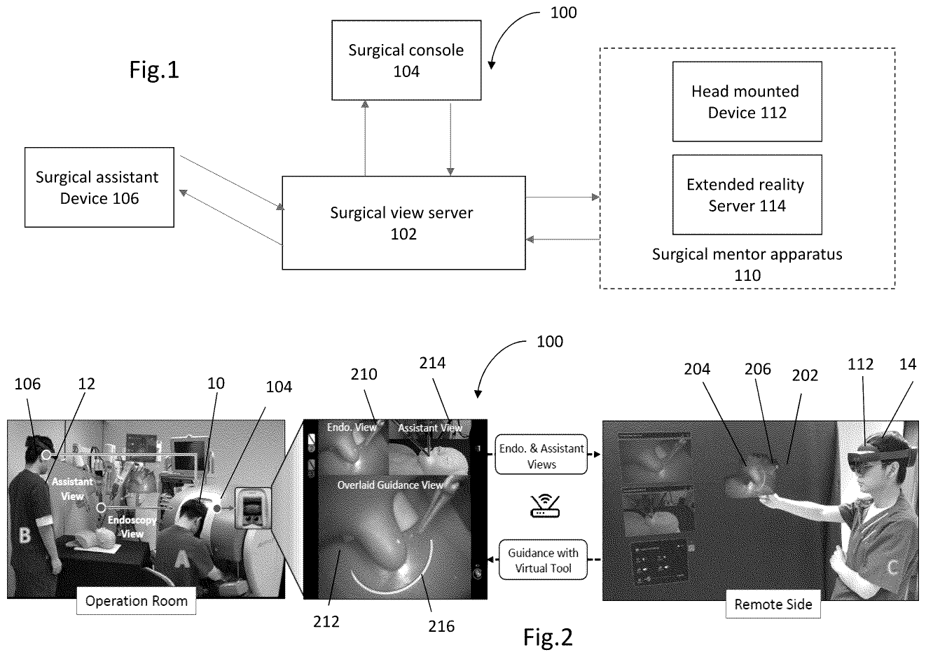

Referring to FIG. 1 and FIG. 2, an embodiment of the present invention is illustrated. FIG. 1 illustrates a schematic diagram of an extended reality system for robotic surgery. The extended reality system 100 for robotic surgery comprising: a surgical view server 102, a surgical console 104 adapted for use by a surgeon, and a surgeon mentor apparatus 110 adapted for use by a surgeon mentor that can mentor a surgeon performing a surgery and a surgical assistant device 106 operatively coupled to the surgical view server 102 and configured to communicate with the surgical view server 102.

The extended reality system provides a system that allows for surgical education and mentoring from one or more remote parties. The system provides an improved user experience and computational efficiency. The system 100 supports 3D scene reconstruction, compression and point cloud transmission addressing technical challenges of real time data processing and communication during surgical training or mentoring.

The surgeon mentor apparatus 110 is arranged remote to the surgical console 102. The surgical view server 102 is configured to wirelessly communicate with the surgical console 104 and surgeon mentor apparatus 110. The surgical console 104, the surgical assistant device 106 and the surgeon mentor apparatus 110 are arranged as clients to the surgical view server 102. The surgical view server 102 is configured to receive a video of a surgical scene from a surgical scope, transmit the video of the surgical scene to the surgeon mentor apparatus. The surgeon mentor apparatus configured to reconstruct surgical scene as a 3D virtual surgical scene, present the 3D virtual surgical scene and transmit surgery guidance received from the surgeon mentor at the surgeon mentor apparatus 110.

The surgical view server 102 is further configured to receive guidance from the surgeon mentor, overlay the guidance from the surgeon mentor onto the surgical scene from the surgical scope, and transmit the overlaid guidance from the surgeon mentor onto the video of the surgical scene for presentation to the surgeon. The surgical console 104 is configured to present the video of the surgical scene with the guidance overlaid onto the video of the surgical scene.

The surgical mentor apparatus 110 comprises a head mounted device 112 and an extended reality server 114. The extended reality server 114 is operatively coupled to the head mounted device 112 and configured to communicate with the head mounted device 112. The head mounted device 112 and the extended reality server 114 are capable of two-way communication with each other. The extended reality server 114 and the head mounted device 112 are arranged in a distributed computing architecture. The head mounted device 112 may function as a client to the extended reality server 114.

The head mounted device 112 may be optical see through head mounted display (OST-HMD). For example, the head mounted device 112 may be a Hololens device. Other extended reality head mounted devices are contemplated.

The head mounted device 112 comprises at least a GPU, sensors e.g., accelerometers etc., optionally a camera, a user interface and other computing hardware capable of allowing 3D visualization. The surgical assistant device 106 may also comprise an optical see through head mounted display (OST-HMD). Alternatively, the surgical assistant device 106 may comprise a head mounted camera.

The extended reality server 114 is configured to perform 3D scene processing and rendering operations to render the 3D virtual surgical scene and transmit the rendered 3D virtual surgical scene to the head mounted device for presentation to a surgeon mentor. Complex processing tasks are offloaded from the head mounted device 112 to the extended reality server 114. The extended reality server is configured to perform image processing, generate point clouds that can be used by the head mounted unit 112 to generate a 3D virtual surgical scene.

The system 100 is advantageous because it allows a remote surgeon mentor to provide guidance to a surgeon performing a surgical process in substantially real time. The guidance from the remote surgeon mentor may be overlaid onto the real-world video from a surgical scope to provide real time guidance to a surgeon.

Another advantage of the system 100 is the integration of data flow between the head mounted device 112 and the extended reality server 114, where the extended reality server 114 is configured to perform complex computations and offload processing from the head mounted device 112 which enhances performance and reduces latency. This enables the system 100 to efficiently render and transmit large scale 3D point clouds allowing for seamless real time interaction in a surgical environment.

The surgical view server 102 is configured to receive a video of a surgical scene from a surgical scope. The surgical scope may be part of the surgical console 104. The surgical scope may be an endoscope or other scope used by the surgeon performing a surgery using the surgical console 104. The scope may include a pair of cameras and may record a surgical scene as a stereo video stream. The surgical view server 102 is configured extract RGB data from the received video and estimate a depth map from the received video. The surgical view server 102 may be adapted to determine a disparity map and estimate a depth map from the disparity map. The surgical view server 102 is configured to combine the RGB data and the depth map into an encoded RGBD data, serialize the RGBD data to generate a stream of RGBD data, and transmit the serialized RGBD data to the surgical mentor apparatus 110. The RGBD data may be a data structure that has combine the RGB data and the depth map data.

The head mounted device 112 e.g., Hololens device is configured to receive the RGBD data and transmit the RGBD data to the extended reality server 114 for processing. The Hololens device 112 may be configured to communicate RGBD data and other data to the extended reality server 114 via a PC-Hololens protocol. The protocol integrates the RGBD data as well as data from the Hololens' built-in sensors, including a front facing camera, and head set pose information for processing. The extending reality server 114 is configured to perform high workload tasks such as 3D scene processing and rendering and returns the results back to the Hololens. The head mounted device 112 may be an extended reality device comprising components that are configured to create and present a virtual reality environment or augmented reality environment or mixed reality environment.

The extended reality server 114 is configured to perform image processing on the received RGBD data, generate a point cloud from the RGBD data, integrated pose information and sensor information and transmit the data back to the head mounted device 112. The head mounted device 112 is further configured to generate a 3D virtual surgical scene based on the point cloud and global coordinate frame and display the 3D virtual surgical scene a user interface. The GPU in the head mounted device 112 (e.g., HoloLens) is configured to decode the results of the real time data from the server 114 to generate and present the 3D virtual surgical scene.

The surgical view server is further configured to receive guidance from the surgeon mentor and overlay the guidance from the surgeon mentor onto the surgical scene from the surgical scope. The guidance may be displayed on the head mounted device 112. The guidance may include gestures from the mentor that are captured by the camera and/or other sensors of the head mounted device 112. The guidance may for example include manipulation of a virtual surgical tool that is presented to the user. The virtual surgical tool may be presented in the 3D virtual surgical scene. The virtual surgical tool may be a 3D virtual tool that is manipulable by the mentor. The surgical scene and surgical tool may be presented in an extended reality or mixed reality environment. The mentor can naturally interact with the virtual tool and indicate cuts or surgical actions that need to be performed on the virtual surgical scene. The actions of the mentor are defined as the guidance.

The server 114 is further configured to transmit the overlaid guidance 216 from the surgeon mentor onto the video of the surgical scene for presentation to the surgeon. The guidance may be indicated as an overlaid line or curve or may be indicated by an overlaid virtual surgical tool. The surgical console is configured to present the video of the surgical scene with the guidance overlaid onto the video of the surgical scene.

The surgical assistant device 106 arranged as a client to the surgical view server 102. The surgical assistant device 106 comprises a camera and is configured to capture video of the assistant's view. The surgical view server 102 is configured to transmit the video of the surgical scene to the surgical assistant, device 106 receive a surgical assistant's view from the surgical assistant device and overlay the surgical assistant's view onto the surgical scene. The view server 102 is further configured to transmit the surgical scene with the overlaid guidance and the overlaid surgical assistant's view to the surgical console for presentation to the surgeon. The surgical console 104 is configured to present the video of the surgical scene with the guidance and the surgical assistant's view overlaid onto the video of the surgical scene.

The system is advantageous because the system's architecture allows multiple users to assist the surgeon. The system is advantageous because it allows for multi user collaboration in real time.

FIG. 2 illustrates an example of a real world set up of the extended reality system 100. As shown in FIG. 2, a surgeon 10 is associated with the surgeon console 104 and uses the surgeon console to perform robotic surgery. The surgeon console includes a user interface e.g. a display or screen that allows a surgeon to view a surgical scene from a scope e.g., two cameras. The surgical assistant 12 (i.e., local assistant) utilizes the surgical assistant device 106 which in the illustrated example is a head mounted display (i.e., head mounted unit) with at least a camera. The remote mentor 14 monitors the surgical procedure and provides guidance through natural hand gestures with built in virtual surgical tools 206 in an extended reality space 202. As shown in FIG. 2, the mentor wears the head mounted device 112 and can remotely monitor the surgery and provide guidance.

As shown in FIG. 2, a surgical scene 20 that is recorded by the surgical scope is presented as a 3D virtual surgical scene 204. The 3D virtual surgical scene 204 may be projected in an extended reality space 202 and allows the mentor to perceive depth due to the 3D nature of the virtual scene. One or more virtual surgical tools i.e., ghost tools 206 may be provided in the extended reality space 202. The tools may occupy the same space as the virtual surgical scene 204. The virtual tools 206 are manipulable by the surgeon mentor 14 using hand gestures. The head mounted device 112 may incorporate an intuitive, hand-based interaction mechanism that utilizes the onboard sensors and camera and pose estimation of the head mounted device 112. The hand gestures are detected by the head mounted device and transmitted server 102. The server 102 is configured to overlay the mentor guidance 212 and the surgical assistant view 214 on to the endoscopy view 210. The endoscopy view with the overlaid views 212, 214 are presented to the surgeon on the surgical console.

The guidance 216 is a motion that is indicative of an ideal cut. The guidance 216 is overlaid as a curved line. In one example, the guidance provided by the surgeon mentor may be in 3D to provide the surgeon with a more realistic perception. For example, the guidance may be a 3D curve or 3D movements to be performed by a tool. Alternatively, the guidance may be indicative of any other surgical functions or operation e.g., suturing, cutting, cauterizing or any other surgical function.

The system 100 is configured to provide a shared extended reality space or extended reality environment. The shared environment allows inputs and interactions from multiple users of the system e.g., the surgeon mentor 14 and the surgical assistant to provide support to the surgeon and improve surgical outcomes. The system allows for real time interaction in the shared extended reality system due to the distributed processing between the server 114 and the head mounted device 112, which reduces latency. Additionally, the architecture of the system provides for efficient synchronized interactions in the shared extended reality space.

In the illustrated example, the surgical view server 102 may be by a computer having an appropriate user interface and computing components. The server 102 may be implemented by any suitable computing architecture, including portable computers, tablet computers, stand-alone Personal Computers (PCs), smart devices, Internet of Things (IOT) devices, edge computing devices, client/server architecture, “dumb” terminal/mainframe architecture, cloud-computing based architecture, or any other appropriate architecture. The computing device (i.e., server 102) may be appropriately programmed to implement the invention.

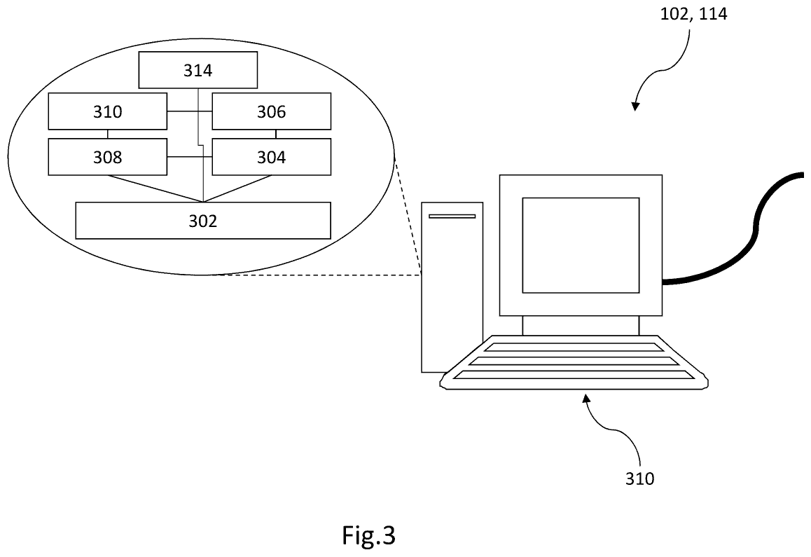

As shown in FIG. 3 there is a shown a schematic diagram of a computer or computing device that is implemented to as the surgical view server 102. The server 102 and server 114 includes suitable components necessary to receive, store and execute appropriate computer instructions. The components may include a processing unit 302, including Central Processing Unit (CPU), Math Co-Processing Unit (Math Processor), Graphic Processing Unit (GPUs) or Tensor processing unit (TPUs) for tensor or multi-dimensional array calculations or manipulation operations, read-only memory (ROM) 304, random access memory (RAM) 306, and input/output devices such as disk drives 308, input devices 310 such as an Ethernet port, a USB port, etc. The server 102 may optionally include a display. The server 102 may include instructions that may be included in ROM 304, RAM 306 or disk drives 108 and may be executed by the processing unit 302. There may be provided a plurality of communication links 312 which may variously connect to one or more computing devices such as a server, personal computers, terminals, wireless or handheld computing devices, Internet of Things (IoT) devices, smart devices, edge computing devices. At least one of a plurality of communications link may be connected to an external computing network through a telephone line or other type of communications link.

The server 102 may include storage devices such as a disk drive 308 which may encompass solid state drives, hard disk drives, optical drives, magnetic tape drives or remote or cloud-based storage devices. The server 102 may use a single disk drive or multiple disk drives, or a remote storage service. The server 102 may also have a suitable operating system which resides on the disk drive or in the ROM of the server 102. The server 102 may further comprise one or more databases adapted to store one or more pieces of data.

The computer or computing apparatus 102 (i.e., server 102) may also provide the necessary computational capabilities to operate or to interface with a machine learning network, such as a neural networks, to provide various functions and outputs. The neural network may be implemented locally, or it may also be accessible or partially accessible via a server or cloud-based service. The machine learning network may also be untrained, partially trained or fully trained, and/or may also be retrained, adapted or updated over time. The computing apparatus may comprise one or more GPUs being operatively coupled to the CPU (i.e., processor). The computing apparatus may comprise additional hardware elements operatively coupled to the CPU and/or the GPU to provide the computing apparatus components needed to implement a machine learning network or machine learning model. The learning network or model may be stored in a memory unit e.g., ROM. In one example the server 102 may implement a machine learning model e.g., an AI model for generating a depth map from disparity maps.

The extended reality server 114 may also be implemented as a computer or computing device as described in relation to FIG. 3. The extended reality server 114 may include the components as shown in FIG. 3. The extended reality server 114 may comprise a PC that comprises a 14 core 2.4 GHZ Intel i9-12900 hk CPU, 32 GB RAM and an NVIDIA RTX 3080Ti GPU. The server 114 may further comprise Vuforia software that is executed by the CPU and GPU. The server 114 may optionally execute a Unity engine. The server 112 may also utilise a Unity3D program to implement the scene of the surgical assistant and remote mentor clients.

The head mounted device 112 may comprise a Hololens device. In one example a Hololens 2 device. The Hololens 2 device may be adapted to communicate with server 114 by a wireless link. Alternatively, a wired connection may be used.

In the system 100 the server 102 directly captures the stereo video from a surgical scope e.g., a laparoscope to predict disparity map and RGB data. The RGB data and disparity maps of the surgical scenario are transmitted to the remote mentor apparatus 110 as a combined data structure (RGBD data). The RGBD data may also be transmitted to the surgical assistant device 106. The extended reality server 114 is configured reconstruct the 3D surgical scene in the head mounted device 112 (i.e., an extended reality device). Experts can naturally use hand-based interaction to grasp the virtual instruments to indicate their suggestions or use the label tool to type and highlight critical anatomy. The guidance from remote mentors is stereo occlusion-aware overlaid on the endoscopic stream 210 and shown in the robot's console 104. Additionally, the assistant's perspective is transmitted to both the console surgeon and the remote mentor, thereby enriching the information in the operating environment.

The surgical console 104 may be a Da Vinci Si surgical robot platform. Other robotic surgery platforms are contemplated. The surgical view server 102 may be equipped with a NVIDIA RTX 3080 unit that is adapted to connect with the surgical console client for real time disparity mapping. The console 104 may be coupled to the server 102 by a wired connection. Alternatively, the console 104 may be coupled via a wireless connection. The surgical assistant device 106 and the surgical mentor apparatus 110 is coupled to the surgical view server 102 by a wireless connection.

To reconstruct the 3D surgical scene stereo matching techniques are utilised to find corresponding pairs between left and right stereo images (Iₗ, Iᵣ). The stereo images are rectified as (Iₗ, Iᵣ) based on the pre-calibrated camera parameters aligning the epipolar lines so that corresponding points lie on the same image row. The server 102 is configured to implement a disparity estimation model that can perform in real time. The model has a running time of 15 FPS. In one example, the disparity estimation model may be HITNet to estimate a disparity map

$${I}_{δ}^{′} .$$

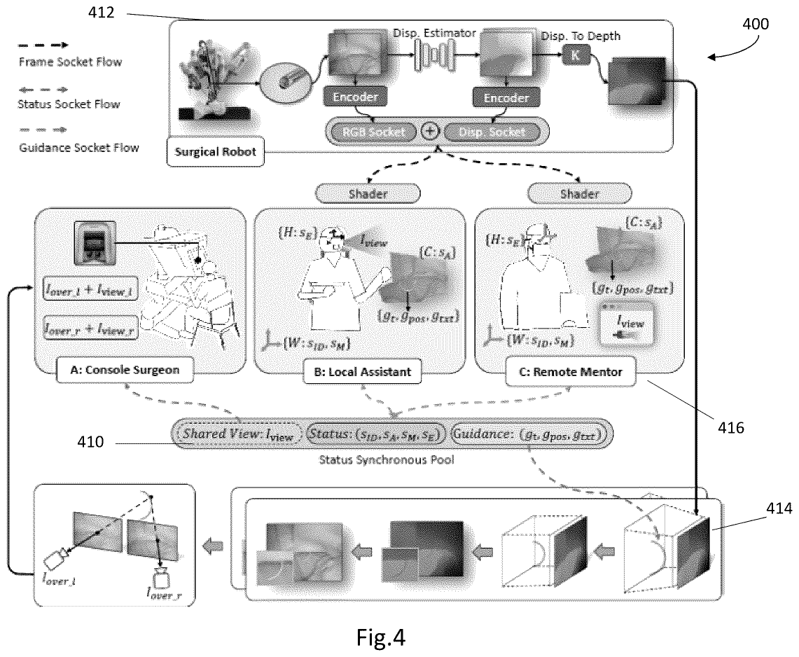

The server 102 may be upgradable to implement new disparity estimation models. FIG. 4 illustrates a system workflow 400 of the extend reality system for robotic surgery.

A RGB-Disp protocol to convert the RGB frame and estimated disparity map into a useable communication socket for streaming the 3D surgical scene information, as shown in 402. RGB data is extracted from video frames. Disparity map (or maps) is extracted by processing the video by applying a disparity map estimator. The estimator may be a machine learning model.

The server 102 or console 104 is configured to utilise a window function Ω(⋅) to constrain the disparity value within range (θ_{inf}, θₛᵤₚ) for excluding outliers. In one example (θ_{inf}=15 px, θₛᵤₚ=64 px) may be predefined. The RGB frame and disparity map are encoded and finally serialize the encoded data as endoscopic streaming. The above operations can be written as following functions:

$$Ω ( p ) = \frac{sgn ( {I}_{δ}^{′} ( p ) - {θ}_{inf} ) - sgn ( {I}_{δ}^{′} ( p ) - {θ}_{sup} )}{2} \qquad ( 1 )$$

$${I}_{δ}^{″} ( p ) = Ω ( p ) \cdot {I}_{δ}^{′} ( p ) , ξ = encode ( {I}_{1}^{′} ) ⊕ encode ( {I}_{δ}^{″} )$$

where p∈ [symbol] ²=(u, v)^{T} denotes the point on the pixel coordinate, f is the focal length, and b is the baseline length. sgn(⋅) indicates the sign function. And ⊕ represents the concatenation operation. ξ means the streaming socket data. A “JPEG” compression is used for the RGB frame. For encoding disparity map, the map is created from

$$[ min ( {I}_{δ}^{″} ) , max ( {I}_{δ}^{″} ) ]$$

scale to [0,255] scale and then do “JPEG” compression. A TCP header may be customised which contains additional information like control signal, and camera parameters for the final endoscopic streaming socket at 414.

A real-time reconstructed point cloud of the laparoscopic scenario is synchronously shown in the client users' extended reality device, and their operation in the extended reality space will also be synchronized at 416. In one example a star topology communication network centre was built on the computing server 102 based on the TCP/IP protocol.

The client devices e.g., the surgeon mentor apparatus 110 and the surgical assistant device 106 may receive the streaming sockets described above. The socket may define the RGBD data i.e., combined into the socket or sockets. The socket may define the combined RGBD data in a serialized and compressed format. The RGBD data is configured for streaming by the client devices 110, 112.

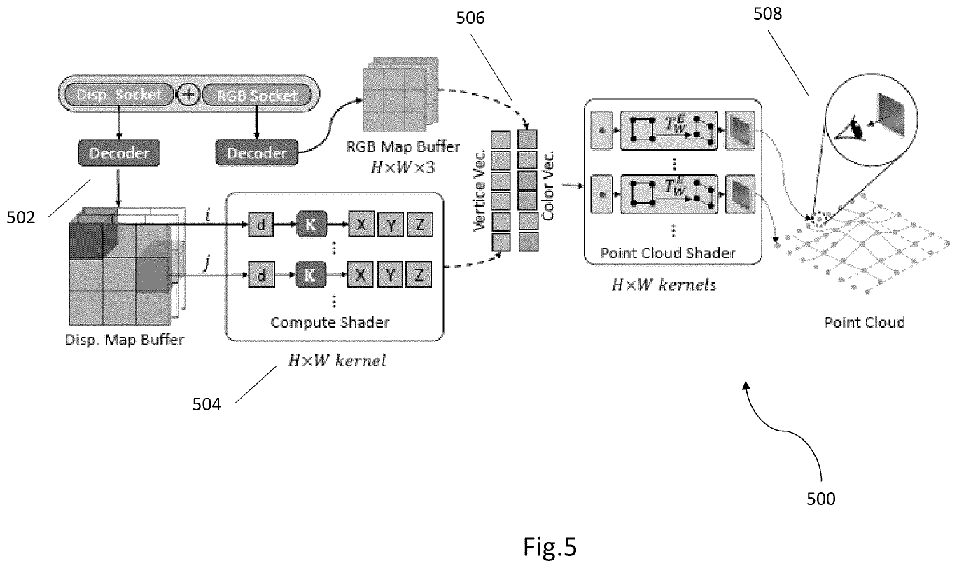

FIG. 5 illustrates a method 500 point cloud computing and rendering in shader. After decoding the socket message, the clients obtain RGB map buffer

$${I}_{l}^{′} ,$$

disparity map buffer

$${I}_{δ}^{″}$$

at 502. Shaders are implemented to render it as the point cloud in OST-HMD for GPU acceleration. As shown in FIG. 5 the pixel index in and pixel vector d of the disparity map are loaded into the compute shader's compute kernel at 50 4, where d is the disparity value. The homogeneous 3D position of a point pₕ=(x, y, z, m)^{T} can be calculated with a projection matrix K, as shown in Eq. 2.

$${p}_{h} = K [ \text{} \begin{matrix}⌊ {i}_{n} / w ⌋ \\ {i}_{n} mod w \\ d \\ 1\end{matrix} \text{} ] , K = [ \text{} \begin{matrix}1 & 0 & 0 & - {c}_{x} \\ 0 & 1 & 0 & - {c}_{y} \\ 0 & 0 & 0 & f \\ 0 & 0 & 1 / b & 0\end{matrix} \text{} ] \qquad ( 2 )$$

where cx, cy, f are the endoscopic camera principle point and focal length, and b is the baseline distance of the stereo camera, w is the width of the disparity map. As shown in FIG. 5, the server 112 is configured to perform dehomogenisation to obtain the final vertex position p=(x/m, y/m, z/m)^{T}. In one example a point cloud shader is used for low computation cost rendering.

The position and colour of each point is first loaded into the vertex shader as a small voxel. In the geometry shader at 506. The point is transmitted into a small square and the normal direction to the user's eye by the matrix

$${T}_{W}^{E} .$$

The matrix coverts Trom the world coordinate {W} to the eye coordinate {E}. The point is finally coloured in the fragment shader as shown in FIG. 5. The additional coordinate transformation

$${( {\dot{p}}^{T} , 1 )}^{T} = {T}_{W}^{H} \cdot {T}_{C}^{W} \cdot {( {p}^{T} , 1 )}^{T}$$

is required to render correctly on the head mounted device 112. The subscript and superscript {H, W, C} of transformation matrix T∈ [symbol] ₃ representatively indicates the head mounted device (i.e., OST-HMD), world and camera coordinate frame at 508. For example,

$${T}_{W}^{H}$$

represents the transformation matrix converting world coordinate to OST-HMD coordinate.

Referring to FIG. 4, the framework for handling multi-port communication utilises a synchronous queue 410. The status synchronous queue 410 ensures synchronization of scenarios and operations between multiple users. Synchronization information is classified into status, guidance, and shared view by their type. Status information {s_{ID}, s_{O}, s_{A}, s_{E}} respectively represents client ID, operation status, point cloud visibility, and shared view button. The guidance information {gₜ, gₚₒₛ gₜₓₜ} indicates guidance type, guidance position, and additional text message. Shared view information {I_{view}} means the view of the camera mounted on the front of the OST-HMD of the surgeon mentor 14 or optionally the surgical assistant 12. State information is used as a control signal for the transmission of guidance and shared view information. For example, s_{ID} is used for client recognition, and s_{A}, s_{E} is used to control the display of guidance and shared view. s_{O} is to address the operation collision problem when multiusers operate at the same time.

The system 100 and the surgeon mentor apparatus 110 is provides an easy to use interface and simple hand gestures to help remote surgeon mentors to provide surgical guidance to surgeons via the head mounted unit 110 using an extended reality space. The system 100 provides a realistic overlay with 3D occlusion perception to fuse guidance from the surgeon mentor apparatus 110 (e.g., the head mounted device 112) with an endoscopic video.

A CAD model of a surgical tool may be loaded into the extended reality server 114 or the server 102. The CAD model is transformed from a camera frustrum space into NDC space with a guidance pose gₚₒₛ and camera parameter K. The server 114 or head mounted unit may be configured to implement a visualisation tool kit (VTK) to implement this transform step. A depth map is calculated using Eq. 2 from a disparity map. The depth map is compared with the depth value of the model in NDC space to obtain an occluded map, which results in the overlay frame. Both left and right augmented images are calculated to produce stereo occlusion aware overlaid images and the 3D perception is generated by binocular disparity. This overlying process may be executed by the server 102. The shared view from the surgical assistant device 106 can be added to the surgeon's view at the console to help identify potential hazards or issues, ensure patient safety and prompt the surgeon 10 to make timely adjustments to improve the outcomes of the surgical procedure.

In the system 100 complex computational tasks are offloaded from the head mounted device 112 to the extended reality server 114. The process of point cloud computing and rendering in a shader as shown in FIG. 5 are processed by the extended reality server 114 rather than in the Hololens device 112. Additionally, the extended reality space rendering processing is executed by the extended reality server 114 and the point cloud data is passed to the head mounted device 112 for rendering as an extended reality space.

The extended reality server 114 is configured to generate a point cloud from the RGBD data and transmit the point cloud data to the head mounted unit 112 to render the 3D virtual space. The server 114 is further configured to generate a virtual surgical tool and transmit the virtual surgical tool to the head mounted device. The head mounted device 112 is configured to present the virtual surgical tool on the 3D virtual surgical scene and detect input gestures from the surgeon mentor. The gestures correspond to movements of the virtual surgical tool, and wherein the movements of the virtual surgical tool define the guidance from the surgeon mentor. The head mounted device 112 is configured to transmit the guidance as movements of a surgical tool to the surgical view server for overlaying the guidance onto the video of the surgical scene. This offloading of processing is known as applying a third-party perspective approach to processing.

The separation of processing i.e., offloading processing to a separate server e.g., a powerful PC like server 114 allows real time depth estimation which provides accurate spatial coordinates estimation of virtual objections. The distributed processing of RGBD data (i.e., RGB data and depth maps) allows for real time rendering of virtual objects reducing latency and improves accuracy and precision of rending virtual objects.

The server 114 may implement Vuforia software or a Vuforia engine. The server 114 utilises Vuforia's localization feature to perform a one-time QR marker or other physical marker localization on the Hololens 112. This allows the server 114 to acquire a spatial correction matrix, effectively anchoring our virtual objects to their correct positions in physical space. By combining the spatial correction matrix with the third-party perspective approach, the server 114 achieves accurate and reliable dynamic environment detection and spatial coordinate estimation, ensuring a seamless and immersive AR experience.

The server 114 may optionally convert the received video from the surgical scope into USB video streams. The server 114 is configured to reduce the video resolution to 480×320 pixels. Subsequently, the server 114 is configured to employ a modified version of the LZ77 algorithm to compress the frames, which were further processed using Huffman coding to optimize the compression ratio and facilitate efficient transmission. The encoded video stream is then decoded upon reception by the Hololens client 112. This dual-stage compression approach markedly mitigates bandwidth demands associated with video transmission while concurrently decreasing memory consumption on the Hololens receiver. Such optimizations are crucial for achieving optimal real-time synchronization between the PC 114 and Hololens 112 platforms.

This integration enables the server 114 to provide a comprehensive and enriched visual experience, combining the real-time video feed from the surgical scope with the virtual objects rendered by the Hololens. The resulting visual assistance offers surgeons a more detailed and accurate view of the surgical site. The synchronized display of both video streams ensures that the virtual objects are aligned with the real-world surgical site, providing a more intuitive and natural guidance system for surgeons.

A PC-Hololens communication protocol was implemented between the extended reality server 114 (PC) and Hololens 112. The communication protocol bridges the Hololens and a PC to address the computational limitations of the HoloLens or other extended reality devices. The protocol integrates data from the Hololens' built-in sensors, including the front-facing camera, depth maps, and headset pose information, and transmits this data to a powerful PC for intensive computation. The server 114 (i.e., PC 114) is configured to perform high-workload tasks, such as 3D scene processing and rendering, and returns the processed results to the Hololens. The Hololens GPU is configured to decode the results for real-time rendering, significantly enhancing the overall system performance and user experience during surgical training.

The Hololens 112 is adapted to render the extended reality environment that includes the 3D virtual surgical scene and one or more virtual surgical tools. Movement or manipulation of the tools based on detected hand gestures may be captured by the Hololens cameras and transmitted for processing to the server 114 (PC 114). The server 114 may process the movements and move the virtual tool accordingly and transmit the data back to the Hololens for rendering.

Conventional systems depend on flat 2D displays. The proposed extended reality system delivers immersive 3D visualization of surgical scenes, significantly enhancing depth perception and spatial awareness. This provides users with a more realistic and comprehensive understanding of the surgical environment. Traditional systems often burden extended reality devices with computational tasks, resulting in hardware limitations and reduced performance. The described system overcomes these challenges by implementing a PC-Hololens communication protocol, where computationally intensive tasks are offloaded to a powerful PC e.g., server 114. This approach allows the extended reality device (e.g., head mounted device 112) to focus on lightweight operations, leading to improved performance, lower latency, and an optimized user experience.

Additionally, the present system it employs an intuitive hand-based interaction method specifically designed for robotic surgical training. By eliminating the need for external tools like touch pens or other devices, it enables seamless, natural user input. While conventional telementoring solutions are typically limited to dual-user interactions—such as between a trainee and a remote expert—the proposed system 100 is capable of supporting multiple users and provides multi-user synchronization, fostering better collaboration and more efficient operations.

The present system 100 is advantageous because ensures efficient handling of large-scale 3D data, including point cloud representations, to optimize performance in extended reality environments for surgical training. The present system 100 utilises a distributed computation network where complex processing is distributed to a separate device to the head mounted extended reality device e.g., Hololens 112. The distributed computation framework is advantageous as it significantly enhances the performance and reduces latency of the extended reality system 100, overcoming hardware limitations of standalone extended reality devices.

The system 100 provides a purely hand-based interaction mechanism within the extended reality environment. This allows users to interact with the system intuitively, eliminating the need for external devices such as touch pens or controllers, making the interaction natural and highly suitable for robotic surgical training. The system 100 is advantageous because it provides a framework that supports multiple users simultaneously. The framework facilitates synchronized operations within a shared extended reality space, making it extendable and adaptable to various surgical training scenarios.

Although not required, the embodiments described with reference to the Figures can be implemented as an application programming interface (API) or as a series of libraries for use by a developer or can be included within another software application, such as a terminal or personal computer operating system or a portable computing device operating system. Generally, as program modules include routines, programs, objects, components and data files assisting in the performance of particular functions, the skilled person will understand that the functionality of the software application may be distributed across a number of routines, objects or components to achieve the same functionality desired herein.

It will also be appreciated that where the methods and systems of the present invention are either wholly implemented by computing system or partly implemented by computing systems then any appropriate computing system architecture may be utilised. This will include stand alone computers, network computers and dedicated hardware devices. Where the terms “computing system” and “computing device” are used, these terms are intended to cover any appropriate arrangement of computer hardware capable of implementing the function described.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Any reference to prior art contained herein is not to be taken as an admission that the information is common general knowledge, unless otherwise indicated.

In its various aspects, embodiments of the invention can be embodied in a computer-implemented process, a machine (such as an electronic device, or a general-purpose computer or other device that provides a platform on which computer programs can be executed), processes performed by these machines, or an article of manufacture.

1. An extended reality system for robotic surgery comprising:
a surgical view server comprising a processor and a memory unit,
a surgical console adapted for use by a surgeon,
a surgeon mentor apparatus adapted for use by a surgeon mentor that can mentor a surgeon performing a surgery,
wherein the surgeon mentor apparatus is remote to the surgical console,
the surgical view server is configured to wirelessly communicate with the surgical console and surgeon mentor apparatus,
the surgical console and the surgeon mentor apparatus are arranged as clients to the surgical view server,
wherein the surgical view server is configured to:
receive a video of a surgical scene from a surgical scope,
transmit the video of the surgical scene to the surgeon mentor apparatus,
wherein the surgeon mentor apparatus configured to:
reconstruct surgical scene as a 3D virtual surgical scene,
present the 3D virtual surgical scene,
transmit surgery guidance received from the surgeon mentor at the surgeon mentor apparatus,
wherein the surgical view server is further configured to:
receive guidance from the surgeon mentor,
overlay the guidance from the surgeon mentor onto the surgical scene from the surgical scope,
transmit the overlaid guidance from the surgeon mentor onto the video of the surgical scene for presentation to the surgeon, and;
wherein the surgical console is configured to:
present the video of the surgical scene with the guidance overlaid onto the video of the surgical scene.

2. The system of claim 1, comprising a surgical assistant device operatively coupled to the surgical view server and configured to communicate with the surgical view server, the surgical assistant device arranged as a client to the surgical view server,
wherein the surgical assistant device comprises a camera and is configured to capture video of the assistant's view,
wherein the surgical view server is configured to:
transmit the video of the surgical scene to the surgical assistant,
receive a surgical assistant's view from the surgical assistant device,
overlay the surgical assistant's view onto the surgical scene,
transmit the surgical scene with the overlaid guidance and the overlaid surgical assistant's view to the surgical console for presentation to the surgeon, and
wherein the surgical console is configured to present the video of the surgical scene with the guidance and the surgical assistant's view overlaid onto the video of the surgical scene.

3. The system of claim 1, wherein the guidance is overlaid in 3D onto the video of the surgical scene.

4. The system of claim 3, wherein the guidance is overlaid onto the video of the surgical scene by stereo occlusion aware overlaying.

5. The system of claim 4, wherein the guidance overlaid onto the video the surgical scene comprises one or more of: a virtual surgical tool and direction of motion of the virtual surgical tool, or an arrow or line indicating a motion of a surgical tool being used by the surgeon.

6. The system of claim 1, wherein the 3D virtual surgical scene corresponds to a surgical scene visible in the video, and the 3D virtual surgical scene is presented on an interface of the surgical mentor apparatus as an extended reality space.

7. The system of claim 2, wherein the surgical view server is configured to:
extract RGB data from the received video,
estimate a depth map from the received video,
combine the RGB data and the depth map into an encoded RGBD data,
serialize the RGBD data to generate a stream of RGBD data,
transmit the serialized RGBD data to the surgical mentor apparatus and the surgical assistant device in real time for 3D reconstruction.

8. The system of claim 7, wherein the surgical assistant device and the surgeon mentor apparatus are configured to stream the serialized RGBD data in real time, and;
reconstruct the 3D virtual surgical scene based on the RGBD data and wherein the 3D virtual surgical scene corresponds to the surgical scene captured in the video, and the 3D virtual surgical scene is updated in real time to correspond with the surgical scene captured in the video.

9. The system of claim 8, wherein the surgical mentor apparatus comprises:
a head mounted device,
an extended reality server operatively coupled to the head mounted device and configured to communicate with the head mounted device,
wherein the extended reality server is configured to perform 3D scene processing and rendering operations to render the 3D virtual surgical scene and transmit the rendered 3D virtual surgical scene to the head mounted device for presentation to a surgeon mentor.

10. The system of claim 9, wherein the extended reality server is configured to:
receive the RGBD data from the surgical view server,
decode the received RGBD data,
render the RGBD data as a point cloud,
each point in the point cloud is plotted on a global coordinate frame,
transmit the point cloud to the head mounted device, and;
the head mounted device is configured to render the 3D virtual surgical scene based on the point cloud and global coordinate frame and display the 3D virtual surgical scene a user interface.

11. An extended reality system for robotic surgery comprising:
a surgical view server comprising a processor and a memory unit,
a surgical console adapted for use by a surgeon,
a surgical assistance device adapted for use by a surgical assistant,
a surgeon mentor apparatus adapted for use by a surgeon mentor that can mentor a surgeon performing a surgery,
wherein the surgeon mentor apparatus is remote to the surgical console,
wherein the surgeon mentor apparatus comprises a head mounted device and an extended reality server configured to communicate with the head mounted device, the extended reality server and the head mounted device are arranged in a distributed computing architecture,
the head mounted device is configured to be function as a client of the extended reality server,
the head mounted device and the
the surgical view server is configured to wirelessly communicate with the surgical console and surgeon mentor apparatus,
the surgical console, the head mounted device, the extended reality server and the surgical assistance device are arranged as clients to the surgical view server,
wherein the surgical view server is configured to:
receive a video of a surgical scene from a surgical scope,
extract RGB data from the received video,
estimate a depth map from the received video,
combine the RGB data and the depth map into an encoded RGBD data,
serialize the RGBD data to generate a stream of RGBD data,
transmit the serialized RGBD data to the surgical mentor apparatus,
wherein the head mounted device is configured to:
receive the RGBD data,
transmit the RGBD data to the extended reality server for processing,
wherein the extended reality server is configured to:
perform image processing on the received RGBD data,
generate a point cloud from the RGBD data,
wherein the head mounted device is further configured to:
generate a 3D virtual surgical scene based on the point cloud and global coordinate frame and display the 3D virtual surgical scene a user interface,
wherein the surgical view server is further configured to:
receive guidance from the surgeon mentor,
overlay the guidance from the surgeon mentor onto the surgical scene from the surgical scope,
transmit the overlaid guidance from the surgeon mentor onto the video of the surgical scene for presentation to the surgeon, and;
wherein the surgical console is configured to:
present the video of the surgical scene with the guidance overlaid onto the video of the surgical scene.

12. The system of claim 11, wherein the surgical assistant device comprises a camera and is configured to capture video of the assistant's view,
wherein the surgical view server is configured to:
transmit the video of the surgical scene to the surgical assistant,
receive a surgical assistant's view from the surgical assistant device,
overlay the surgical assistant's view onto the surgical scene,
transmit the surgical scene with the overlaid guidance and the overlaid surgical assistant's view to the surgical console for presentation to the surgeon, and
wherein the surgical console is configured to present the video of the surgical scene with the guidance and the surgical assistant's view overlaid onto the video of the surgical scene.

13. The system of claim 11, wherein the guidance is overlaid in 3D onto the video of the surgical scene and wherein the guidance is overlaid onto the video of the surgical scene by stereo occlusion aware overlaying.

14. The system of claim 13, the surgical assistant device and the surgeon mentor apparatus are configured to stream the serialized RGBD data in real time, and; reconstruct the 3D virtual surgical scene based on the RGBD data and wherein the 3D virtual surgical scene corresponds to the surgical scene captured in the video, and the 3D virtual surgical scene is updated in real time to correspond with the surgical scene captured in the video.

15. The system of claim 14, wherein the surgical assistant device comprises optical see through head mounted display that comprises a camera.

16. The system of claim 14, wherein the surgical view server is configured to:
build a synchronous queue to ensure synchronization of scenarios and operations between the head mounted device and the surgical assistant device,
wherein the synchronization information is classified into status, guidance and a shared view information,
wherein status information represents client device ID, operation status, point cloud visibility and a shared view button,
wherein the guidance information indicates guidance type, guidance position and additional text message,
wherein the share view information relates to a view from a camera of the surgical assistant device and/or the head mounted device, and;
wherein the synchronous queue is configured to reduce the operation collision problem.

17. The system of claim 16, wherein the 3D virtual surgical scene corresponds to a surgical scene visible in the video, and the 3D virtual surgical scene is presented as a hologram on an interface of the head mounted device in an extended reality space.

18. The system of claim 17, wherein the extended reality server is configured to:
receive the RGBD data from the surgical view server,
decode the received RGBD data,
render the RGBD data as a point cloud,
each point in the point cloud is plotted on a global coordinate frame, and;
transmit the point cloud to the head mounted device,
wherein the extended reality server is further configured to:
generate a virtual surgical tool,
transmit the virtual surgical tool to the head mounted device,
wherein the head mounted device is configured to:
present the virtual surgical tool on the 3D virtual surgical scene,
detect input gestures from the surgeon mentor, wherein the gestures correspond to movements of the virtual surgical tool, and wherein the movements of the virtual surgical tool define the guidance from the surgeon mentor,
transmit the guidance as movements of a surgical tool to the surgical view server for overlaying the guidance onto the video of the surgical scene.

19. The system of claim 18, wherein the head mounted device is a Hololens device that is adapted to generate an extended reality interface where the virtual surgical tool can be overlaid on the 3D virtual surgical scene.

20. The system of claim 1, wherein the system is configured to render over 300,000 points with a latency of less than 250 ms.
